(19) United States
(12) Patent Application Publication
(10) Pub. No.: US 2020/0331973 A1
(43) Pub. Date: Oct. 22, 2020

(54) NUCLEIC ACID ENCODING CRM197 AND PROCESS FOR IMPROVED EXPRESSION THEREOF

(71) Applicant: LORVEN BIOLOGICS PRIVATE LIMITED, Andhra Pradesh (IN)

(72) Inventors: Lavanya PUPPALA, Andhra Pradesh (IN); Venkataramana MUDILI, Andhra Pradesh (IN)

(21) Appl. No.: 16/643,243

(22) PCT Filed: Aug. 29, 2018
(86) PCT No.: PCT/IB2018/056586

§ 371 Date: Feb. 28, 2020

(30) Foreign Application Priority Data
Sep. 1, 2017 (IN) 201741031140

(51) Int. Cl. C07K 14/34; C12N 15/75; C12P 21/02

(52) U.S. Cl. CPC C07K 14/34; C12P 21/02; C12N 15/75

(57) ABSTRACT

The present invention provides for a nucleic acid encoding CRM₁₉₇ protein and process for improved expression of CRM₁₉₇ protein. The invention represents an advancement in the field of genetic engineering and discloses a modified nucleic acid for achieving optimum expression of CRM₁₉₇ protein in a heterologous host. The invention also discloses vectors carrying the modified nucleic acid and recombinant host cells carrying the vectors. The invention also discloses the process for producing a recombinant host cell, process for production of the recombinant protein and an improved down streaming process.

### CROSS REFERENCE TO RELATED APPLICATION AND CLAIM OF PRIORITY

The present application claims priority from Indian Patent Application No. 201741031140 filed on Sep. 1, 2017, the entire contents of which are hereby incorporated by reference.

### FIELD OF INVENTION

The present invention is directed to the field of genetic engineering. More particularly, the invention is directed to a biologically active recombinant CRM₁₉₇ protein and methods for obtaining improved expression of biologically active CRM₁₉₇ protein in Bacillus subtilis by strain engineering.

### BACKGROUND OF THE INVENTION

CRM₁₉₇ is a nontoxic mutant of diphtheria toxin. Native CRM₁₉₇ is composed of a single polypeptide chain of 535 amino acids (58 kD) and nicked by cellular protease like furin to give two fragments which are linked by disulfide bridges. The fragments wok synchronously to penetrate the host cell. CRM₁₉₇ has an alteration of 52^{nd} Gly to Glu and exhibits neither ADP ribosylation activity nor toxicity to cells. This single base change results in an amino acid substitution (glutamic acid for glycine) and this point mutation results in a significant loss of toxicity. This mutation causes intrinsic flexibility of the active-site loop in front of the NAD-binding site and reduces the ability of CRM₁₉₇ to bind NAD and eliminates toxic properties of DT. CRM₁₉₇ exhibits no enzymatic activity and is immunologically indistinguishable from wild-type Diphteria toxin. CRM₁₉₇ competitively inhibits binding of HB-EGF to HB-EGF receptor.

Many conjugated polysaccharide vaccines like like Menveo®, Menjugate®, Prevnar®, and HibTITER® use CRM₁₉₇ as a carrier protein.

CRM₁₉₇ has been found to be an ideal carrier for conjugate vaccines against encapsulated bacteria. The carrier protein is covalently linked to poorly immunogenic and T-cell-independent capsular polysaccharides, thus creating T-cell-dependent conjugate antigens that are highly immunogenic in infants. Vaccines containing CRM₁₉₇ as a carrier protein have been successfully used to immunize millions of children. Therefore, there is a huge demand of CRM₁₉₇ protein which can be used for production of cheap, affordable and effective vaccines, especially for third world countries.

CRM₁₉₇ has been prepared by expression in Corynebacterium diphtheriae and other microorganisms in the prior art. The wild-type diphtheria toxin may be obtained from toxin producing strains available from a variety of publicly accessible sources. Various strategies have been explored to produce CRM₁₉₇ on a commercial scale by recombinant DNA technology using a wide range of heterologous hosts.

However, there are major disadvantages in heterologous expression of CRM₁₉₇. The high levels of expression lead to a precipitation of recombinant protein in the bacterial cytoplasm as “inclusion bodies”. In many cases proteins remain an insoluble precipitate that can only be released into solution by using strong chaotropic agents. Therefore, the purification of heterologous or recombinant protein is disadvantageous as the desired protein has to be purified from endogenous host cell.

The outer cell membrane of most Gram-negative bacteria, e.g. E. coli, contains lipopolysaccharides (LPS), generally referred to as endotoxins, which are pyrogenic in humans and other mammals. These endotoxins complicate product purification, because the end-product should be completely endotoxin-free.

Due to these reasons, the yield of biologically active CRM₁₉₇ obtained by heterologous expression are extremely low due to degradation, improper folding, inefficient translocation and high downstream processing costs.

Therefore, the present invention contemplates to overcome the challenges of the prior art by expressing an engineered nucleic acid encoding CRM₁₉₇ in a heterologous host and providing methods for producing large amounts of CRM₁₉₇ in a biologically active form.

The present invention further contemplates that by using Bacillus expression system that produces and ideally secretes CRM₁₉₇ into the culture medium, the system itself enables an initial purification of the manufactured protein due to the absence of contaminating endogenous bacterial protein and other macromolecules. Thus, apart from solving the technical problem of low yield, the present invention holds economic significance as it would directly affect the cost of conjugate vaccines using CRM₁₉₇, making the vaccines more affordable and widely accessible.

### SUMMARY OF THE INVENTION

The present invention relates to a modified nucleic acid encoding a fusion protein in which CRM₁₉₇ protein is operably fused to alpha amylase signal peptide of B. amyloliquefaciens and the process for obtaining a high yield of the recombinant CRM₁₉₇ protein.

In one aspect, the invention provides a modified nucleic acid encoding a fusion protein in which the nucleic acid encoding CRM₁₉₇ protein has been operably fused to the alpha amylase signal peptide of Bacillus amyloliquefaciens.

In another aspect, the invention provides a recombinant vector containing a modified nucleic acid encoding CRM₁₉₇ protein fused to alpha amylase signal peptide of B. amyloliquefaciens.

In yet another aspect, the invention provides a recombinant host cell which can optimally express CRM₁₉₇ protein.

In yet another aspect, the invention provides a modified fusion protein in which CRM₁₉₇ protein has been fused to the alpha amylase signal peptide of Bacillus amyloliquefaciens.

In a further aspect, the invention provides an improved process for production for obtaining a high yield of CRM₁₉₇ protein.

### BRIEF DESCRIPTION OF THE DRAWINGS

The features of the present disclosure will become fully apparent from the following description taken in conjunction with the accompanying figures. With the understanding that the figures depict only several embodiments in accordance with the disclosure and are not to be considered limiting of its scope, the disclosure will be described further through use of the accompanying figures.

FIG. 1 is the vector map of recombinant plasmid pBR322 which shows the gene construction for expression of recombinant fusion protein.

FIG. 2 depicts SDS-PAGE of cell fractions obtained from the recombinant strains and control strains.

FIG. 3 depicts the results of Western Blot analysis for identification of the protein obtained from recombinant strain.

FIG. 4 depicts the results of nuclease activity assay.

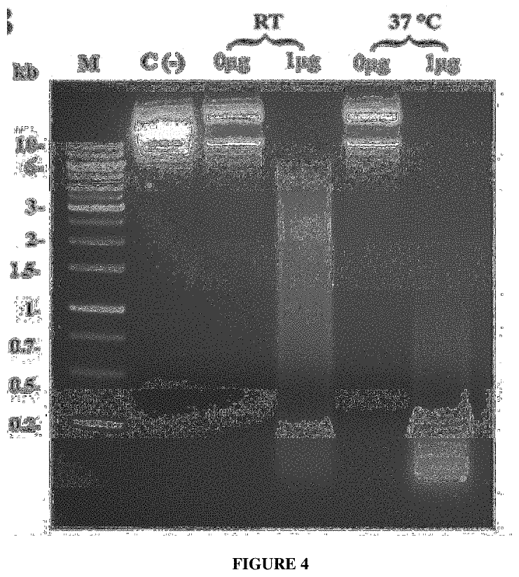

### DETAILED DESCRIPTION OF THE INVENTION

The present invention discloses a modified nucleic acid encoding CRM₁₉₇ protein which is optimally expressed in a heterologous host.

The invention contemplates that the modified nucleic acid would have better expression in a heterologous host leading to better yield of the protein.

The invention also contemplates an improved downstream processing method for recovery of biologically active CRM₁₉₇.

The invention contemplates a multidimensional approach for achieving a high rate of expression in a heterologous host. Primarily, the invention contemplates that fusing the nucleotide sequence encoding alpha amylase signal peptide of Bacillus amyloliquefaciens with the sequence encoding CRM₁₉₇ leads to more efficient translocation of CRM₁₉₇ protein across the cell membrane. This approach coupled with engineering the nucleotide sequence of the native gene encoding CRM₁₉₇ to match the preferred codon system of the host cell gives a greater efficiency in protein expression.

Unless otherwise defined herein, scientific and technical terms used in connection with the present disclosure shall have the meanings that are commonly understood by those of ordinary skill in the art. Further, unless otherwise required by context, singular terms shall include the plural and plural terms shall include the singular as is considered appropriate to the context and/or application. The various singular/plural permutations may be expressly set forth herein for the sake of clarity. Generally, nomenclatures used in connection with, and techniques of biotechnology, fermentation technology, genetic engineering and recombinant DNA technology described herein are those well-known and commonly used in the art. Certain references and other documents cited are expressly incorporated herein by reference. In case of conflict, the present specification, including definitions, will control. The materials, methods, figures and examples are illustrative only and not intended to be limiting.

Furthermore, the methods, preparation and use of the modified nucleic acid encoding the CRM₁₉₇ employ, unless otherwise indicated, conventional techniques in recombinant DNA technology, fermentation technology and related fields. These techniques, their principles, and requirements are explained in the literature and known to a person skilled in the art.

Before the method of generating the modified nucleic acid encoding the CRM₁₉₇, vectors, recombinant hosts, methods of downstream processing and other embodiments of the present disclosure are disclosed and described, it is to be understood that the terminologies used herein are for the purpose of describing particular embodiments only and are not intended to be limiting.

It must be noted that, as used in the specification and the appended claims, the singular forms “a,” “an” and “the” include plural referents unless the context clearly dictates otherwise.

As used herein, the term “comprises” or “comprising” is generally used in the sense to include, that is to say permitting the presence of one or more features or components.

As used herein, the term “disclosure” or “present disclosure” as used herein is a non-limiting term and is not intended to refer to any single embodiment of the particular disclosure but encompasses all possible embodiments as described in the specification and the claims.

As used herein, the term “CRM” or “CRM₁₉₇” refers to a nontoxic mutant of diphtheria toxin.

As used herein, the term “gene” refers to a nucleic acid fragment corresponding to specific amino acid sequence that expresses a specific protein with regulatory sequences. “Native gene” or “wild type gene” refers to a gene as found in nature with its own regulatory sequences.

As used herein, the term “promoter” refers to a region of DNA that initiates transcription of a particular gene. Promoters are located near the transcription start sites of genes, on the same strand and upstream on the DNA capable of controlling the expression of a coding sequence or functional RNA which can be native, derived or synthetic. Some promoters are called constitutive as they are active in all circumstances in the cell, while others are called inducible as they are regulated and become active in response to specific stimuli.

The term “inducible promoter” refers the promoters that are induced by the presence or absence of biotic or abiotic and chemical or physical factors. Inducible promoters are a very powerful tool in genetic engineering because the expression of genes operably linked to them can be turned on or off at certain stages of development or growth of an organism or in a particular tissue or cells.

As used herein, the term “gene expression”, refers to the process by which information from a gene is used in the synthesis of a functional gene product. These products are often proteins, but in non-protein coding genes such as transfer RNA (tRNA) or small nuclear RNA (snRNA) genes, the product is a functional RNA.

As used herein, the term “transformation” as used herein, refers to the transfer of a nucleic acid fragment into a host organism either in the form of plasmid or integrated stably to the chromosome of the host organisms resulting in genetically stable inheritance. A cloning vector is a small piece of DNA, mostly a plasmid, that can be stably maintained in an organism, and into which a foreign DNA fragment can be inserted for cloning or transformation purposes.

The term “host cell” includes an individual cell or cell culture which can be, or has been, a recipient for the subject of expression constructs. Host cells include progeny of a single host cell. Host cell can be any expression host including prokaryotic cell such as but not limited to Bacillus subtilis, Escherichia coli, Pseudomonas putida, Corynebacterium glutamicum or eukaryotic system, such as, but not limited to Saccharomyces cerevisiae, Pichia pastoris, Hansenula polymorpha.

The term “recombinant strain” refers to a host cell which has been transfected or transformed with the expression constructs or vectors of this invention.

The term “expression cassette” denotes a gene sequence used for cloning in expression vectors or in to integration vectors or integrated in to coding or noncoding regions of chromosome of the host cell in a single or multiple copy numbers, where the expression cassette directs the host cell's machinery to make RNA and protein encoded by the expression cassette.

The term “expression construct” is used here to refer to a functional unit that is built in a vector for the purpose of expressing recombinant proteins/peptides, when introduced into an appropriate host cell, can be transcribed and translated into a fusion protein which is displayed on the cell wall.

The term “modified nucleic acid” or “modified nucleotide sequence” is used to refer to an artificially synthesized nucleic acid in which the gene encoding CRM₁₉₇ protein has been operably fused to the alpha amylase signal peptide of B. amyloliquefaciens.

Although disclosure and exemplification has been provided by way of illustrations and examples for the purpose of clarity and understanding, it is apparent to a person skilled in the art that various changes and modifications can be practiced without departing from the spirit or scope of the disclosure. Accordingly, the foregoing descriptions and examples should not be construed as limiting the scope of the present disclosure.

The present invention discloses a modified nucleic acid encoding CRM₁₉₇ protein in which the gene encoding CRM₁₉₇ protein has been operably fused to alpha amylase signal peptide of B. amyloliquefaciens and having optimal expression levels in heterologous hosts. In a preferred embodiment, the nucleic acid is represented by SEQ ID NO: 1.

The present disclosure also relates to a polypeptide encoded by the nucleic acid sequence as in SEQ ID NO: 1 or any variant thereof, wherein the polypeptide is CRM₁₉₇ protein fused to the alpha amylase signal peptide of Bacillus amyloliquefaciens.

In another embodiment, the present disclosure discloses suitable vectors comprising the modified nucleic acid for optimal expression of CRM₁₉₇ protein in a heterologous host. In yet another embodiment, the vector of the disclosure is an expression vector which can be conveniently subjected to recombinant DNA procedures. The choice of vector will often depend on the host cell into which it is to be introduced. Thus, the vector could be an autonomously replicating vector, i.e. a vector which exists as an extra-chromosomal entity, the replication of which is independent of chromosomal replication, e.g. a plasmid. Alternatively, the vector could be one which, when introduced into a host cell, is integrated into the host cell genome, in part or in its entirety, and replicated together with the chromosomes into which it has been integrated.

In another embodiment, the vector is preferably an expression vector in which the DNA sequence encoding the CRM₁₉₇ is operably linked to additional segments required for transcription of the DNA. The term, “operably linked” indicates that the segments are arranged so that they function in concert for their intended purposes, e.g. transcription initiates in some promoter and proceeds through the DNA sequence coding for the enzyme.

Preferably, the gene can be cloned into any Bacillus subtilis expression vectors known in the art. In a preferred embodiment, the vector is a pBR322.

Any suitable promoter can be used. In a preferred embodiment, an inducible promoter Pgrac is used. The cloned gene sequences can be confirmed by restriction digestion or nucleotide sequencing. In a preferred embodiment, the vector pBR322 having Pgrac promoter has been used.

In another embodiment, the host cell into which the DNA construct or the recombinant vector of the disclosure is introduced may be any cell which can produce the present enzyme and includes bacteria, yeast, any other microorganism, a mammalian cell, plant cell or any cell culture of said category.

In a preferred embodiment, the host-cell is a bacterial cell selected from a group comprising Bacillus subtilis, Escherichia coli, Lactococcus lactis, Bacillus megaterium, Pseudomonas putida and Corynebacterium glutamicum or the host cell is a eukaryotic cell selected from a group comprising Saccharomyces cerevisiae, Pichia pastoris and Hansenula polymorpha or any host known in the art for expression of heterologous proteins using T7 promoter-based vectors for expression.

In a preferred embodiment, the host-cell is Bacillus subtilis WB800N. Commercially available Bacillus subtilis WB800N was used in the preferred embodiment of the invention. In the preferred embodiment, the host cell is defective in the expression of multiple proteases namely nprE, aprE, epr, bpr, mpr, ble, nprB, bsr, vpr, wprA, hrc A or a combination thereof.

In another embodiment, the expression level of the gene was measured by quantifying the amount of recombinant protein. Bradford protein assay was performed for quantifying the protein present in the sample. The disclosure provides enhanced expression of the recombinant CRM₁₉₇ using the recombinant Bacillus subtilis which may range upto 15 g/L.

In another embodiment, the process for production of CRM₁₉₇ protein is provided. In a preferred embodiment, the process of production includes the steps of culturing host cells transformed with a vector comprising a modified nucleic acid of SEQ ID NO: 1 in a suitable culture medium, isolating recombinant CRM₁₉₇ protein from the cell culture and purifying recombinant CRM₁₉₇ protein.

In another embodiment, the process of culturing host cells transformed with a vector comprising the modified nucleic acid comprises of continuously adding a carbon source, continuously adding IPTG and stopping the culture process when OD₆₀₀ of 70 to 110 is reached. In a preferred embodiment, the feed rate of the carbon source is between 2.25 to 7.5 g/L/hr, preferably 6.7 g/L/hr. In a preferred embodiment, the carbon source is glucose.

In another embodiment, the method for isolating recombinant CRM₁₉₇ protein is given. In a preferred embodiment, the process of isolating the CRM₁₉₇ protein involves the process of adding 10% to 14% PEG at pH of 5.0-8.5, loading a first anion exchange resin material with the CRM₁₉₇ in a loading buffer in the absence or low concentration of divalent cations, optionally followed by washing, eluting the CRM₁₉₇ with an eluent comprising a divalent cation and a counter-anion to form an eluate containing the CRM₁₉₇. Commercially available PEG2000, PEG4000, PEG6000 or PEG20000 may be used in the said invention.

In another embodiment, the process for purifying recombinant CRM₁₉₇ using hydrophobic interaction chromatography is given. In a preferred embodiment, the process comprises the steps of:
a. contacting a sample comprising the protein of interest and at least one impurity, to a hydrophobic interaction chromatography (HIC) media, in the presence of a load buffer such that (i) a portion of the protein of interest binds to the HIC media and (ii) a substantial portion of the at least one impurity binds to the HIC media;
b. collecting a flow through fraction comprising the protein of interest unbound to the HIC media; and
c. washing the HIC media with a wash buffer that is substantially the same as the load buffer such that a substantial portion of the protein of interest bound to the HIC media is released from the media; and collecting a wash fraction comprising the protein of interest released from the HIC media, wherein each of the flow through and wash fractions comprise the protein of interest and have a reduced level of the at least one impurity.

### Source and Geographical Origin of Biological Material

Nucleic acid (SEQ ID NO:1) was synthesized artificially by Genscript, USA, a commercial gene synthesis service provider. The vector pBR322 was obtained from Thermo Fisher Scientific, USA. The host cell Bacillus subtilis WB800N was obtained from MoBiTec GmbH, Germany.

### EXAMPLES

The following examples particularly describe the manner in which the invention is to be performed. But the embodiments disclosed herein do not limit the scope of the invention in any manner.

### Example 1

### Gene Construction for Expression of Recombinant CRM₁₉₇ Protein in Bacillus subtilis

Gene encoding for CRM₁₉₇ protein was fused with the signal peptide of Bacillus amyloliquefaciens. The modified gene contains a modified open reading frame encoding for CRM protein fused to alpha amylase signal peptide of Bacillus amyloliquefaciens. The preferred codons for expression in Bacillus subtilis has been used in place of rare codons.

The sequence of the modified open reading frame encoding for CRM₁₉₇ protein fused signal peptide of Bacillus amyloliquefaciens is represented by SEQ ID NO: 1. This modified open reading frame has been artificially synthesized by using the sequence for CRM₁₉₇ protein of Cornybacterium diptheriae and the signal peptide of Bacillus amyloliquefaciens.

The plasmid used in the process was pBR322. The recombinant plasmid contains the open reading frame and an inducible Pgrac promoter.

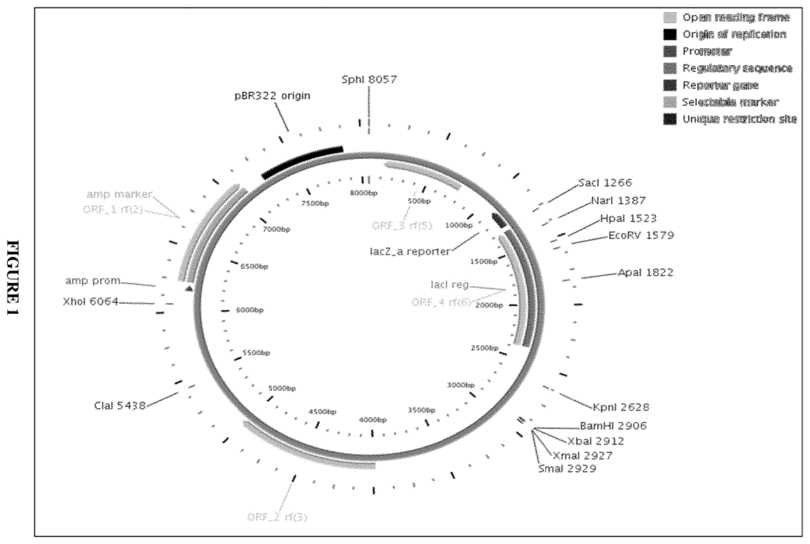

The modified sequence encoding for the recombinant protein was cloned in to pBR322 expression vector. The vector map is represented in FIG. 1.

### Example 2

### Polynucleotide Sequence for Expression of CRM₁₉₇ Protein and Corresponding Polypeptide Sequence

The recombinant protein obtained by translating the gene encoding for CRM₁₉₇ protein fused with the signal peptide of Bacillus amyloliquefaciens is represented by SEQ ID NO: 2. The recombinant protein is CRM₁₉₇ protein of Cornybacterium diptheriae fused with signal peptide of Bacillus amyloliquefaciens.

### Example 3

### Development of Recombinant Host Cell by Transformation with Gene Construct

Recombinant pBR322 plasmids as described in foregoing example carrying the gene for CRM₁₉₇ sequence fused to alpha amylase signal peptide of Bacillus amyloliquefaciens represented by SEQ ID NO: 1 were used.

Host cells were electroporated with the plasmids as described in foregoing example and resuspended in growth medium with trace minerals. The cultures were incubated at 30° C. with shaking for 48 hours. 10 μL of each of the seed cultures were transferred into triplicate test tubes, each tube containing 5 ml of growth medium supplemented with trace elements and incubated for 24 hours.

IPTG was added to each well to a final concentration of 1%. 24 hrs after induction, supernatant samples were frozen for later processing.

### Example 4

### Large-Scale Expression of CRM₁₉₇ Protein

Recombinant CRM₁₉₇ protein was produced in the recombinant host cell in 35-liter fermenter. Cultures were grown in 35-liter fermenter containing nutrient broth, as semi synthetic medium.

Culture conditions were maintained at 37° C. and pH 7.0 through the addition of sodium hydroxide. Dissolved oxygen was maintained at 20% though increases in agitation and flow of sparged air and oxygen into the fermenter. Glucose was used as a carbon source delivered to the culture throughout the fermentation to maintain excess levels. The feed rate of the glucose was kept between 2.25 to 7.5 g/L/hr. The best results were obtained at a feed rate of carbon at around 6.7 g/L/hr.

These conditions were maintained until a target cell culture density at OD₆₀₀ was reached, at which time IPTG was added for induction of CRM₁₉₇ production. Cell density at induction varies from A₆₀₀ of 70 to 110 absorbance units (AU). IPTG concentrations were varied in the range from 0.01 to 1.0 mM and pH was varied from 6 to 7.5. The temperature was maintained between 20° C. to 35° C. After 16-30 hours, the culture from each bioreactor was harvested by centrifugation and the culture supernatant was frozen at −80° C. Samples were analyzed by Reverse Phase HPLC and Western blot analysis for product formation.

Multiple fermentation conditions were evaluated resulting in top CRM₁₉₇ expression as determined by Reverse Phase HPLC. The identities of the induced proteins were confirmed by Western blot analysis using a diphtheria toxin specific antibody.

Yields from large-scale fermentation cultures were found to be typically higher than those obtained in the small HTP cultures. Based on the HCD expression data above, large-scale fermentation yields up to 15 g/L were obtained.

### Example 5

### Isolation of Recombinant CRM₁₉₇

The recombinant protein was isolated from the culture by precipitating the protein by addition of 10% to 14% PEG at pH 5.0-8.5. PEG2000 was used for the purpose. After precipitation, the precipitate was subjected to anion exchange chromatography using standard protocol for isolation.

The process included loading an anion exchange resin material with the CRM₁₉₇ with a loading buffer at low concentration of divalent cations.

It was followed by an optional step of washing. Any anion exchange resin material like diethylaminoethane, dimethylaminoethane, trimethylaminoethyl, polyethyleneimine, quaternary aminoalkyl, quaternary aminoethane and quaternary ammonium can be used. The final step includes eluting the CRM₁₉₇ with an eluent comprising a divalent cation and a counter-anion.

The loading buffer and/or washing buffer, has a pH that is at least 0.5 pH units lower than the pH of the eluent of step. Further, the pH was maintained between 7.0 to 7.4, for a case where an optional wash step was performed and between 5.9 to 6.1 in case where the optional wash step is not performed.

### Example 6

### Purification of Recombinant CRM₁₉₇

Hydrophobic interaction chromatography using standard protocols was used to further purify the recombinant protein. Sepharose Fast Flow HIC platform, manufactured by GE Life Science was used for HIC chromatography. A standard aromatic hydrophobic interaction chromatography (HIC) medium sold under the tradename of PHENYL SEPHAROSE® used in the process is composed of cross-linked, 6% agarose beads modified with standard aromatic phenyl groups via uncharged, chemically-stable ether linkages. Two levels of ligand substitution degree help to find the optimal selectivity and binding capacity for a given application.

The eluent obtained in the aforesaid example was purified by contacting a sample comprising the protein of interest and at least one impurity, to a hydrophobic interaction chromatography (HIC) media, in the presence of a load buffer such that the portion of the protein of interest binds to the HIC media and a substantial portion of the at least one impurity binds to the HIC media.

It was followed by collecting a flow through fraction comprising the protein of interest unbound to the HIC media, washing the HIC media with a wash buffer that is substantially the same as the load buffer such that a substantial portion of the protein of interest bound to the HIC media is released from the media and collecting a wash fraction comprising the protein of interest released from the HIC media, wherein each of the flow through and wash fractions comprise the protein of interest and have a reduced level of the at least one impurity.

### Example 7

### SDS-PAGE

Analysis of CRM₁₉₇ by SDS-PAGE which was purified by one precipitation and two steps of chromatography. The gel was stained by silver stain to visualize the bands.

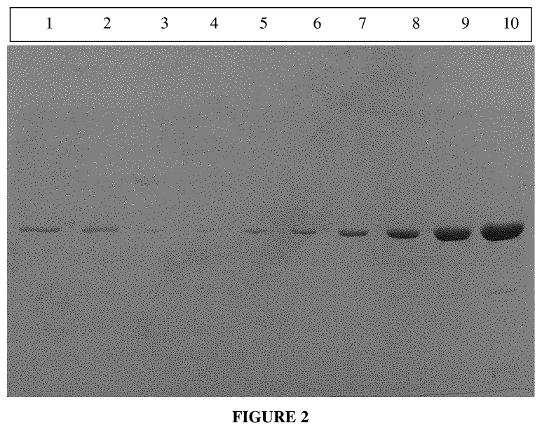

An aliquot of cell culture was collected at different time points and the cell lysates were subjected to SDS-PAGE. The results of SDS-PAGE are depicted in FIG. 2.

For SDS-PAGE, the CRM₁₉₇ protein produced by recombinant Bacillus subtilis cells were collected. The recombinant protein was subjected to SDS-PAGE using standard protocols.

Standard CRM₁₉₇ was put in the first two lanes and in rest of the lanes CRM₁₉₇ purified by standard aromatic hydrophobic interaction chromatography (HIC) medium sold under the tradename of PHENYL SEPHAROSE® were placed.

The details of the CRM₁₉₇ protein obtained in each lane is elaborated in the following table:

TABLE 1
Details of the CRM₁₉₇ protein in each lane

| Lane | Details |
|---|---|
| 1 | Merck Standard - 500 ng |
| 2 | C7 Standard - 500 ng |
| 3 | Recombinant CRM₁₉₇ purified by 0.1 μL |
| | PHENYL SEPHAROSE ® |
| 4 | Recombinant CRM₁₉₇ purified by 0.2 μL |
| | PHENYL SEPHAROSE ® |
| 5 | Recombinant CRM₁₉₇ purified by 0.4 μL |
| | PHENYL SEPHAROSE ® |
| 6 | Recombinant CRM₁₉₇ purified by 0.8 μL |
| | PHENYL SEPHAROSE ® |
| 7 | Recombinant CRM₁₉₇ purified by 1.6 μL |
| | PHENYL SEPHAROSE ® |
| 8 | Recombinant CRM₁₉₇ purified by 3.2 μL |
| | PHENYL SEPHAROSE ® |
| 9 | Recombinant CRM₁₉₇ purified by 6.4 μL |
| | PHENYL SEPHAROSE ® |
| 10 | Recombinant CRM₁₉₇ purified by 12.8 μL |
| | PHENYL SEPHAROSE ® |

It was confirmed that highly purified CRM₁₉₇ protein was obtained after subjecting the culture supernatant to HIC chromatography.

### Example 8

### Western Blot Analysis

The purified CRM₁₉₇ protein was subjected to western blot analysis. Purified CRM₁₉₇ was transferred to nitrocellulose membrane and immunoanalysis was done using anti-diphtheria toxin antibody (Mouse monoclonal [3B6] supplied by Abcam) which specifically reacts with CRM₁₉₇ protein specifically and does not react with free A or B subunits of Diphtheria toxin.

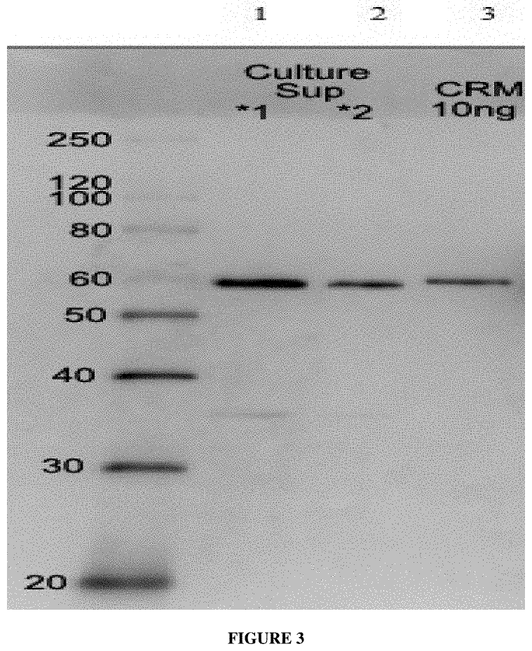

The results of the Western blot are represented in FIG. 3. Protein present in Lane 1 and Lane 2 exactly matches with the CRM₁₉₇ standard protein.

The purified protein showed strong immunogenic response and antisera were collected and purified by chromatography. This result shows that the protein having CRM₁₉₇ activity has been produced by the recombinant cells.

### Example 9

### Nuclease Activity Determination

Recombinant cells were resuspended in nuclease lysis buffer (40 mM Tris-HCl, pH 6.8, 100 mM NaCl, 0.1% SDS, 1% Triton X-100) and clarified by low speed centrifugation (1000×g, 10 min.). Tenfold dilutions were made in nuclease reaction cocktail buffer (100 mM Tris-HCl, pH 8.8, 10 mM CaCl₂, 0.1% NP40) and boiled for 1 minute. Reaction products were electrophoresed on 0.8% agarose gels and DNA was visualized by ethidium bromide staining.

The results of the experiment are depicted in FIG. 3. The results depict that the CRM₁₉₇ protein is in right conformation and biologically active.

### Example 10

### Yield Determination

The amount of recombinant protein was consequently quantified. Quantification was done by employing standard Bradford assay protocol. The results of protein quantification assays reveal that the recombinant protein in sample is present in the ranged between 0.1 mg/L to 15 g/L.

A wide range of dilutions starting from 1, 1:10, 1:100, 1:1000 were prepared and standard Bradford assay protocol was followed. The absorbance was measured at A595 of the samples and standards against the reagent blank between 2 min and 1 h after mixing. The 100-μg standard should give an A595 value was also measured.

In a range of experiments conducted for yield determination, the yield of CRM₁₉₇ was found to be about 0.1 mg/L, about 0.6 g/L, about 0.7 g/L, about 0.8 g/L, about 0.9 g/L, about 1 g/L, about 1.5 g/L, about 2 g/L, about 2.5 g/L, about 3 g/L, about 3.5 g/L, about 4 g/L, about 4.5 g/L, about 5 g/L, about 5.5g/L, about 6 g/L, about 6.5 g/L, about 7 g/L, about 7.5 g/L, about 8 g/L, about 8.5 g/L, about 9 g/L, about 9.5 g/L, about 10 g/L, about 10.5 g/L, about 1 1 g/L, about 15 g/L.

The following table illustrates the best yield of CRM₁₉₇ obtained from the recombinant strain:

TABLE 2
Yield of CRM₁₉₇

| Batch | Feed Rate | Yield |
|---|---|---|
| Number | (g/L/hr) | (g/L) |
| B32 | 6.0 g/L/hr | 9.5 |
| B33 | 6.5 g/L/hr | 10 |
| B34 | 6.7 g/L/hr | 15 |
| B35 | 7.0 g/L/hr | 11 |
| B36 | 7.5 g/L/hr | 10.5 |

This result exhibits that the recombinant cells are capable of expressing a very high amount of protein.

1. A modified nucleic acid comprising the nucleotide sequence of SEQ ID NO: 1, wherein the nucleic acid encodes CRM₁₉₇ protein in a host cell.

2. A vector comprising the nucleic acid as claimed in 1, wherein the nucleic acid is operably linked to a promoter.

3. The vector as claimed in claim 2, wherein the vector is pBR322 and the promoter is Pgrac.

4. A recombinant prokaryotic host cell comprising the vector as claimed in claim 2.

5. The recombinant prokaryotic host cell as claimed in claim 4, wherein the host cell is Bacillus subtilis WB800N.

6. A modified polypeptide comprising a CRM₁₉₇ protein fused to signal peptide of Bacillus amyloliquefaciens, wherein the modified polypeptide comprises the amino acid sequence of SEQ ID NO: 2.

7. A process for producing a recombinant host cell capable of expressing a polypeptide as claimed in 6 comprising the steps of:
a. synthesizing a modified nucleic acid comprising the nucleotide sequence of SEQ ID NO: 1;
b. constructing a recombinant pBR322 vector harbouring the nucleic acid of SEQ ID NO: 1, wherein the nucleic acid is operably linked to a Pgrac promoter; and
c. transforming a Bacillus subtilis WB800N host cell with the recombinant pBR322 vector to obtain a recombinant host cell.

8. A process for production of recombinant CRM₁₉₇ protein, said process comprising the steps of:
a. culturing recombinant host cell as claimed in claim 5 in a suitable culture medium, wherein the step of culturing host cells comprises the steps of:
i. adding continuously a carbon source, wherein the feed rate of the carbon source is between 2.25 to 7.5 g/L/hr;
ii. adding continuously IPTG to the culture for 6 to 9 hrs to achieve OD₆₀₀ of 70 to 110; and
iii. harvesting the recombinant protein from the culture after about 6 to about 9 hrs after commencement of inducer addition.
b. isolating the recombinant CRM₁₉₇ protein from the cell culture; and
c. purifying the recombinant CRM₁₉₇ protein.

9. The process for production of CRM₁₉₇ protein as claimed in claim 8, wherein the step of isolating recombinant CRM₁₉₇ protein comprises the steps of:
a. precipitating the CRM₁₉₇ protein by addition of 10% to 14% polyethylene glycol (PEG) at pH of 5.0-8.5;
b. loading the precipitated CRM₁₉₇ onto an anion exchange resin charged with divalent cations in a loading buffer having pH in the range of 5.5 to 7.5; and
c. eluting the anion exchange resin with an eluent to obtain purified CRM₁₉₇ protein.

10. The process for production of CRM₁₉₇ protein as claimed in claim 9, further comprising after said step (b) and prior to said step (c), the step of washing the anion exchange resin with a washing buffer, wherein the pH of the loading buffer and the washing buffer is 0.5 units lower than the pH of the eluant and the pH of the loading buffer ranges between 7.0 to 7.4.

11. The process for production of CRM₁₉₇ protein as claimed in claim 9, wherein the anion exchange resin material is selected from a group comprising of diethylaminoethane, dimethylaminoethane, trimethylaminoethyl, polyethyleneimine, quaternary aminoalkyl, quaternary aminoethane and quaternary ammonium.

12. The process for production of CRM₁₉₇ protein as claimed in claim 8, wherein the method for purifying recombinant CRM₁₉₇ protein comprises the steps of:
a. contacting a sample comprising the CRM₁₉₇ protein with a hydrophobic interaction chromatography medium in the presence of a load buffer such that a portion of the protein of interest binds to the hydrophobic interaction chromatography medium and a substantial portion of the at least one impurity binds to HIC media;
b. collecting a flow through fraction comprising the protein of interest unbound to the HIC media;
c. washing the hydrophobic interaction chromatography medium with a wash buffer such that a substantial portion of CRM₁₉₇ bound to the HIC media is released from the media; and
d. collecting a wash fraction comprising CRM₁₉₇ protein released from the hydrophobic interaction chromatography medium.


SEQUENCE LISTING (2 sequences)

SEQ ID NO: 1 - DNA, 1698 bp, Artificial Sequence
  Nucleotide sequence encoding CRM197 fused to a nucleotide sequence encoding signal peptide of B. amyloliquefaciens.
atgattcagaaacgcaaacgcaccgtgagctttcgcctggtgctgatgtgcaccctgctgtttgtgagcctgccgattaccaaaaccagcgcgggcgcggatgatgtggtggatagcagcaaaagctttgtgatggaaaactttagcagctatcatggcaccaaaccgggctatgtggatagcattcagaaaggcattcagaaaccgaaaagcggcacccagggcaactatgatgatgattggaaagaattttatagcaccgataacaaatatgatgcggcgggctatagcgtggataacgaaaacccgctgagcggcaaagcgggcggcgtggtgaaagtgacctatccgggcctgaccaaagtgctggcgctgaaagtggataacgcggaaaccattaaaaaagaactgggcctgagcctgaccgaaccgctgatggaacaggtgggcaccgaagaatttattaaacgctttggcgatggcgcgagccgcgtggtgctgagcctgccgtttgcggaaggcagcagcagcgtggaatatattaacaactgggaacaggcgaaagcgctgagcgtggaactggaaattaactttgaaacccgcggcaaacgcggccaggatgcgatgtatgaatatatggcgcaggcgtgcgcgggcaaccgcgtgcgccgcagcgtgggcagcagcctgagctgcattaacctggattgggatgtgattcgcgataaaaccaaaaccaaaattgaaagcctgaaagaacatggcccgattaaaaacaaaatgagcgaaagcccgaacaaaaccgtgagcgaagaaaaagcgaaacagtatctggaagaatttcatcagaccgcgctggaacatccggaactgagcgaactgaaaaccgtgaccggcaccaacccggtgtttgcgggcgcgaactatgcggcgtgggcggtgaacgtggcgcaggtgattgatagcgaaaccgcggataacctggaaaaaaccaccgcggcgctgagcattctgccgggcattggcagcgtgatgggcattgcggatggcgcggtgcatcataacaccgaagaaattgtggcgcagagcattgcgctgagcagcctgatggtggcgcaggcgattccgctggtgggcgaactggtggatattggctttgcggcgtataactttgtggaaagcattattaacctgtttcaggtggtgcataacagctataaccgcccggcgtatagcccgggccataaaacccagccgtttctgcatgatggctatgcggtgagctggaacaccgtggaagatagcattattcgcaccggctttcagggcgaaagcggccatgatattaaaattaccgcggaaaacaccccgctgccgattgcgggcgtgctgctgccgaccattccgggcaaactggatgtgaacaaaagcaaaacccatattagcgtgaacggccgcaaaattcgcatgcgctgccgcgcgattgatggcgatgtgaccttttgccgcccgaaaagcccggtgtatgtgggcaacggcgtgcatgcgaacctgcatgtggcgtttcatcgcagcagcagcgaaaaaattcatagcaacgaaattagcagcgatagcattggcgtgctgggctatcagaaaaccgtggatcataccaaagtgaacagcaaactgagcctgttttttgaaattaaaagc

SEQ ID NO: 2 - protein, 566 aa, Artificial Sequence
  CRM197 of Corynebacterium diphtheriae fused to signal peptide of Bacillus amyloliquefaciens
MIQKRKRTVSFRLVLMCTLLFVSLPITKTSAGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS